(19) United States
(12) Patent Application Publication
(10) Pub. No.: US 2018/0225991 A1
(43) Pub. Date: Aug. 9, 2018

(54) TRAINING APPARATUS FOR SURGICAL PROCEDURES AND TRAINING SYSTEM FOR LAPAROSCOPIC PROCEDURES

(71) Applicants: Miguel Angelo Pedroso, Itu (BR); Mauro de Souza Leite Pinho, Joinville (BR); Thiago Magalhães Bramante, Votorantim (BR); Dyego Sá Benevenuto, Rio de Janeiro (BR)

(72) Inventors: Miguel Angelo Pedroso, Itu (BR); Mauro de Souza Leite Pinho, Joinville (BR); Thiago Magalhães Bramante, Votorantim (BR); Dyego Sá Benevenuto, Rio de Janeiro (BR)

(21) Appl. No.: 15/751,229

(22) PCT Filed: Aug. 9, 2016
(86) PCT No.: PCT/BR2016/050188

§ 371 Date: Feb. 8, 2018

(30) Foreign Application Priority Data
Aug. 11, 2015 (BR) 202015019194-0
Apr. 6, 2016 (BR) 102016007625-0

(51) Int. Cl. G09B 23/28

(52) U.S. Cl. CPC G09B 23/285; A61B 1/04

(57) ABSTRACT

An apparatus and a system for surgical procedure training, allowing the user to simulate real surgical procedures, in addition to allowing simple assembly and disassembly thereof. Specifically, the apparatus of the present invention has a first region, a second region, and a third region implemented in a single material, interconnected by means of points of articulation that allow folding of the material, and thereby assembling the apparatus in question. Further, a support region is arranged, and it is implemented in the same material as the other regions, or individually, subsequently connected. In addition, the regions have fastening means for connection to one another. The present invention lies within the fields of medicine and education.

### STATEMENT OF RELATED APPLICATIONS

The application is the US PCT National Phase of International Application No. PCT/BR2016/050188 having an International Filing Date of 9 Aug. 2016, which claims priority on Brazil Patent Application No. 20 2015 019194-0 having a filing date of 11 Aug. 2015 and Brazil Patent Application No. 10 2016 007625-0 having a filing date of 6 Apr. 2016.

### BACKGROUND OF THE INVENTION

### Technical Field

The present invention describes an apparatus and a system for surgical procedure training, allowing the user to simulate real surgical procedures, in addition to allowing simple assembly and disassembly thereof. The present invention lies within the fields of medicine and education.

### Prior Art

With the advancement of medicine, several technologies regarding surgical techniques are developed in order to facilitate the work of professionals, and primarily avoiding placing the patient's life at risk. One technique applied today is called laparoscopy, which is a type of minimally invasive surgery.

Minimally invasive surgery methods are performed so that there is a small incision in the patient, where surgical devices called laparoscopes are introduced. Such devices consist of a cylindrical instrument, such as a scope, which transmits images from the patient's interior to a monitor where the surgeon can monitor the development thereof. Thus, the surgeon uses optics, tweezers, clamps, suction tubes or more instruments necessary for performing the surgical act, introducing them into the patient, and, through the video transmitted by the laparoscope, monitors the movements that are being performed inside the patient.

Therefore, such technique requires attention from the professional who performs the surgery, since there is a need for proper handling of the instruments, where a wrong movement may cause irreparable damages to the patient. To that end, some types of simulators are being developed for assisting and training these professionals who perform the surgical procedure.

However, the currently shown simulators comprise high dimensions and weight, in addition to needing fixed and specific locations for the professional to practice laparoscopic techniques. Furthermore, such simulators do not allow the professional to carry them in a simple way. Moreover, these current simulators require a set of elements and components that make up such simulators, i.e., the professional must assemble several parts in order to be able to carry out the training.

Further, different surgical procedures have different plans for incision and operation, and the existing training equipment have the limitation of having only one angle of operation, making it difficult to carry out surgical procedure training. That is, current simulators are available for a particular type of surgery, thus, if a professional needs training on different types of surgical procedures, he/she is forced to use another simulator.

While searching for the prior art in the scientific and patent literature, the following documents have been found covering with the topic:

Document U.S. D699,297 discloses an equipment for training professionals in laparoscopic surgery comprising isolated parts being interconnected by means of hinges, so that the equipment is compact to the user. Such solution, however, comprises high manufacturing costs, since it is a rigid material with a need for hinges for interconnecting the parts. In addition, U.S. D699,297 provides for the application of this type of simulator only for practicing a certain type of surgical procedure, thus preventing the professional from being free to practice other surgical specialties and limiting the application thereof.

Document MU 8702124 describes a simulator container for laparoscopic surgeries showing several faces with varying angles and holes for introducing the surgical elements, thus allowing the user to practice the desired procedure. Such solution, however, has a hinge in the upper region, suggesting a need to assemble different parts, making it difficult for the user to assemble it, in addition to making it difficult to transport it to other locations. Further, such solution features the use of a micro-camera, which must comprise its own lighting source, thus causing the user to be forced to acquire said object, since the container shown is isolated from external lighting. Nevertheless, the solution shown by document MU8702124 requires the user to have a specific monitor to receive the type of video signal coming from the micro-cam era.

Document US 2012/082970 discloses a surgical procedure training equipment containing a top and a bottom, which are joined in order to be transported and, when the equipment is put into use, both parts are separated in the assembly thereof by means of hinges or any other element for this purpose, thus offering room for the user to practice surgical procedures. However, the solution shown in the document requires the user to assemble the parts by inserting an additional element to lock both in their respective positions, in addition to requiring the user to assemble all the apparatuses that are necessary for the training, such as, for example, the holes where the surgical elements are inserted.

Document U.S. Pat. No. 6,659,776 discloses a laparoscopic procedure training apparatus comprising holes for insertion of a laparoscopic instrument and an internally coupled camera for user training video transmission. Such a solution, however, requires the user to have a monitor adapted to receive the video signal coming from the camera, in addition to not allowing several angles for training, thus precluding the professional from being free to practice other surgical specialties, and limiting the application thereof.

Thus, from what is apparent from the searched literature, no documents have been found anticipating or suggesting the teachings of the present invention, such that the solution proposed herein has novelty and an inventive step in view of the prior art.

### BRIEF SUMMARY OF THE INVENTION

The present invention has the purpose of solving the problems encountered in the prior art by developing an equipment for practicing and training surgical procedures, wherein said equipment is easy to assemble and transport, since it is implemented in continuous sheet material and comprises its regions separated by articulation points formed in the material itself. Further, the solution of the present invention comprises aperture regions to allow ambient lighting inside the equipment, allowing the user to carry out the practice without the need for additional lighting elements. In addition, the apparatus of the present invention comprises regions for training from various angles in order to enable the user to practice several different types of surgical procedures.

In a first aspect, the present invention provides a surgical procedure training apparatus comprising:
a. a first region (1);
b. a second region (2);
c. a third region (3);
d. a support region (4);
   wherein,

said apparatus is implemented in continuous sheet material comprising points of articulation between: the first region (1) and the second region (2); and the second region (2) and the third region (3);

said points of articulation are of the same nature as the sheet material itself;

the second region (2) comprises fastening means for connecting the support region (4);

all said regions comprise fastening means for interconnecting one another.

In a second aspect, the present invention features a laparoscopic procedure training system comprising:
a. a surgical procedure training apparatus;
b. trocars;
c. an electronic device having an image capture element;
d. a laparoscopic instrument; and
e. a training element;

wherein,

said surgical procedure training apparatus is provided with apertures for introducing medical equipment and apertures for placing an image capture element;

the laparoscopic instrument and the trocar are associated and introduced into the aperture for introducing the medical equipment of the apparatus;

the training element is inserted inside the apparatus;

the electronic device is associated with the apparatus so that the capture element is associated with the aperture for placing the image capture element.

Accordingly, the common inventive concept to all claimed protection contexts relates to a surgical procedure training equipment composed of at least three regions, which are separated by points of articulation of the same nature as the very material to which the regions are implemented, so that said material is flexible and allows said regions to be connected to one another by folding the material, thereby forming a type of box for surgical procedure training. In addition, said regions comprise engaging means that are adapted so that it is possible to effectuate said connections between the regions.

These and other aspects of the invention shall be immediately appreciated by persons skilled in the art and by companies with interest in the segment, and shall be described in sufficient detail for reproduction thereof in the description that follows.

### BRIEF DESCRIPTION OF THE DRAWINGS

With the purpose of better defining and clarifying the content of the present patent application, the following figures are shown:

FIG. 1 shows a top view of a first embodiment of the disassembled apparatus of the present invention.

FIG. 2 shows a detailed view of the first region (1) of said embodiment of the disassembled apparatus of the present invention.

FIG. 3 shows a detailed view of the second region (2) of said embodiment of the disassembled apparatus of the present invention.

FIG. 4 shows a detailed view of the third region (3) of said embodiment of the disassembled apparatus of the present invention.

FIG. 5 shows a detailed view of the support region (4) of said embodiment of the disassembled apparatus of the present invention.

FIG. 6 shows a top view of a second embodiment of the disassembled apparatus of the present invention.

FIG. 7 shows a detailed view of the first region (1) of said embodiment of the disassembled apparatus of the present invention.

FIG. 8 shows a detailed view of the second region (2) of said embodiment of the disassembled apparatus of the present invention.

FIG. 9 shows a detailed view of the third region (3) of said embodiment of the disassembled apparatus of the present invention.

FIG. 10 shows a detailed view of the support region (4) of said embodiment of the disassembled apparatus of the present invention.

FIG. 11 shows a top view of a third embodiment of the disassembled apparatus of the present invention.

FIG. 12 shows a front perspective view of the third embodiment of the assembled apparatus of the present invention.

FIG. 13 shows a rear perspective view of the third embodiment of the assembled apparatus of the present invention.

FIG. 14 shows a front view of the third embodiment of the assembled apparatus of the present invention, omitting the support region (4).

FIG. 15 shows a front view of a variation of the third embodiment of the assembled apparatus of the present invention, omitting the support region (4).

### DETAILED DESCRIPTION OF PREFERRED EMBODIMENTS

The following descriptions are shown only by way of example and non-limiting of the scope of the invention, and shall enable a clearer understanding of the subject of the present patent application.

In order to provide a way for users to practice surgical procedures in a single location, in addition to provide a way that is easily accessible and transported by an user, the present invention, in a first aspect, discloses a surgical procedure training apparatus comprising a first region 1, a second region 2, a third region 3 and a support region 4, such that, generally, said regions may both allow introduction of surgical elements and provide structural support for the apparatus.

The present invention apparatus can be found in at least two different manners, assembled or disassembled, so that, when disassembled, the regions are not connected to each other, where it is used in this manner to allow transportation or storage thereof; on the other hand, when shown in assembled form, the regions are engaged and the apparatus is ready to the user perform the surgical procedure training. Such functionality is provided because said apparatus is implemented in a continuous sheet material, wherein it comprises points of articulation, which are responsible for interconnecting the first region 1 to the second region 2 and the second region 2 to the third region 3. For the purposes of detailing the subject of the present invention, continuous sheet material means a single object, a single material, which may be folded, i.e., more specifically, the apparatus of the present invention is made in a single piece, requiring no external fastening elements for the interconnection of its parts.

Further, the abovementioned points of articulation are formed of the same nature as the continuous sheet material itself, i.e., are implemented within the very single piece from which the apparatus is formed. In one embodiment, the points of articulation are formed due to the fact that the material is foldable, wherein when the material is folded for assembling the apparatus, these points of articulation are formed, thus allowing full flexibility so that the regions of the apparatus are able to be connected.

In one embodiment, the continuous sheet material of the present invention is any type of material that allows folding movement, forming the points of articulation between the regions and not easily wearing out with a single assembly, so that the user may perform the assembly and disassembly steps without breakage or rupture thereof. In a more preferred embodiment, the material of the present invention is of the reinforced cardboard type, or pasteboard type.

In order to be able to implement the apparatus of the present invention in a single piece, thus allowing assembly and disassembly thereof, all regions comprise fastening means which are also formed of the same nature as the material itself. Thus, for the purposes of the present invention, said fastening means are any elements that are formed within the sheet material, and may have different geometries, such as the “male and female” type, where they are connected to each other, thus allowing assembly of the apparatus with the connection of the regions. In one embodiment, the fastening means are of the opening and projection type, wherein an opening is defined by a cut, or the removal of a small portion from part of the apparatus in question. Projections are defined as a relief, or a flap, formed by an outline during the cutting of the piece that forms the apparatus.

Further, the present apparatus comprises at least one structural support flap which is arranged in the first region 1, the second region 2 or the third region 3 by means of points of articulation. Said support flap, as well as the other elements, is also formed in the continuous sheet material itself, so as to show greater stability for assembly of the apparatus, thus enabling the user to carry out the training more steadily, similarly to what is practiced in a real surgery. In the apparatus of the present invention, said flap is implemented in one of the regions so as to connect to at least one other region, and for purposes of exemplification, the support flap is implemented in the second region 2 and connects to the third region 3, wherein this example of assembling the apparatus is not limiting, so that the support flap may be implemented in any of the regions and, therefore, connect with any of the regions.

Thus, in order to connect said support flap to the required regions, fastening means were formed in the support flap, being of the same type as the one comprised in the regions, as already defined above.

In one embodiment, the first region 1 comprises at least one aperture for introducing medical equipment, so that said aperture is disposed at a specific angle which allows the user to carry out the surgical procedure training by simulating a real surgery.

In one embodiment, the second region 2 comprises at least one aperture for introducing medical equipment, so that said aperture is disposed at a specific angle which allows the user to carry out the surgical procedure training by simulating a real surgery. Further, in said embodiment, the second region 2 comprises fastening means for connection with the support region 4, so that this connection is reinforced, thus allowing to place the necessary elements in said support region 4 without damaging the structure of the apparatus. In addition, the second region 2 comprises an aperture for image capture.

In one embodiment, the support region 4 is adapted to receive an electronic device provided with an image capture element. Furthermore, said support region 4 is provided with fastening means for connection with the second region 2, so as to comprise at least one projection perpendicular to its base, i.e., to its own construction. More specifically, said projection is disposed in the direction of the force applied by the electronic device, while said device is positioned in the support region 4 when the apparatus is assembled, wherein such arrangement is provided in order to provide greater stability and greater resistance against the weight of the electronic device when it is positioned in the support region 4, without compromising the structure of the apparatus and preventing the connection between the support region 4 and the second region 2 from yielding. In another embodiment, the support region 4 is individually implemented, not being implemented in the same continuous sheet material as the other regions; however, it has the same type of material used for the other regions. In another embodiment, the support region 4 is implemented in the same continuous sheet material as the apparatus, being interconnected to any of the regions by means of the points of articulation in the material itself.

Additionally, in one embodiment, the second region 2 comprises apertures for insertion of medical equipment at rest, i.e., when the user is not using the medical equipment for practicing the procedure, the latter may be inserted into said apertures, so as to be stored and available to the user when it is needed. Usually during a training, the user has four medical equipment, however, only two may be used at the same time by the user, thus, the apertures mentioned herein are arranged in order to allow the user to store equipment that is not in use.

The third region 3 comprised in the apparatus of the present invention may be used for various purposes and implemented in a number of different ways in order to suit the purpose for which the apparatus is used, as well as the geometry of the apparatus when assembled. In one embodiment, the third region 3 is a base region of the apparatus, serving for structural support, ensuring that the user may practice the surgical procedure more safely. In another embodiment, the third region 3 comprises sectioning and is divided into at least two portions, which are interconnected by points of articulation implemented in the sheet material itself as defined above, wherein said arrangement allows the improvement of the structural support of the apparatus. In a further embodiment, the third region 3 is divided into two parts and comprises, in one of them, at least one aperture for introducing medical equipment, so as to allow the user to have another angle to proceed with the desired surgical procedure training.

The apparatus of the present invention further comprises at least one aperture to allow the incidence of ambient lighting, wherein said aperture is implemented in any one of the regions of the apparatus, the structural support flap, or the rear of the apparatus, where the latter is formed when the apparatus is assembled, said regions having a specific geometry and, in the assembly thereof, the apparatus has a rear part with an aperture allowing the incidence of ambient lighting. Such a point is necessary to allow the user to visualize a training element when practicing the surgical procedure, without using an additional light source, or to have a light-emitting device.

In one embodiment, the apparatus of the present invention is used for laparoscopic procedure training.

In one embodiment, the aperture for introducing medical equipment is adapted for laparoscopy surgical procedure training, such as, for example, endoscopic laparoscopy, gynecological laparoscopy, transanal laparoscopy, or the like.

Thus, as may be noted, the present invention provides an easily mountable and transportable apparatus comprising various angles in its regions which allow the user to practice various types of surgical procedures in single equipment, in addition to comprising apertures to allow the incidence of ambient lighting. Further, the present apparatus is implemented entirely in foldable material, being the main part implemented in a single piece.

In a second aspect, the present invention provides a laparoscopic procedure training system comprising the above-defined apparatus, trocars, electronic device having an image capture element, laparoscopic instrument and training element. Said system is arranged to enable a user to perform laparoscopic procedure training using the apparatus of the present invention associated with the optional basic elements that enable operation thereof.

The trocar of the system, in one embodiment, is a conventional one, wherein it is an element that is inserted into a patient during a laparoscopic surgery, and the laparoscopic instruments are inserted into this element so that the surgeon may perform all the necessary steps for the procedure. In the present invention, the trocar is inserted into the aperture for surgical procedures of the apparatus in question and the laparoscopic instrument is associated with the trocar. This association allows simulating a real environment for surgery of this specialty. However, the apparatus may also be used without the use of the trocar element.

Thus, in order to proceed with the practice of the desired procedure, the user inserts a training element into the apparatus, wherein this training element is a PAD that simulates the inner tissue for practicing sutures. Thereby, the user inserts the training elements into the apparatus and starts the training.

Further, in order to simulate a laparoscopic surgery environment, the user places an electronic device in the support region 4 of the apparatus, so that the image capture element of the device is placed in the aperture for placing the image capture element comprised in the second region 2 of the apparatus. That is, accordingly, the electronic device is capable of capturing images internally to the apparatus through said aperture, where images of the training element are specifically captured and shown on the display of the device. For the purposes of the present invention, the electronic device is any device capable of capturing images and displaying them on a display, in addition to being able to be placed in the support region 4 of the apparatus, such as, for example, a tablet or an iPad®, but not limited thereto, so that the camera lens is placed in the aperture for placing the image capture element comprised in the second region 2, and the captured images from the training element are transmitted to the display of said tablet or iPad® itself.

The system of the present invention is proposed in order to show the elements that are associated with the apparatus so as to allow proper surgical procedure to be practiced, thus simulating the reality of laparoscopic surgery. However, as is apparent from its definition, the apparatus does not require these elements for its complete operation.

The examples shown herein are intended to only exemplify some of the many ways of carrying out the invention, without, however, limiting the scope thereof. Further, other embodiments of implementing the apparatus of the present invention may utilize a combination of the features shown in different examples, thus not restricting the features to the cited example.

### Example 1

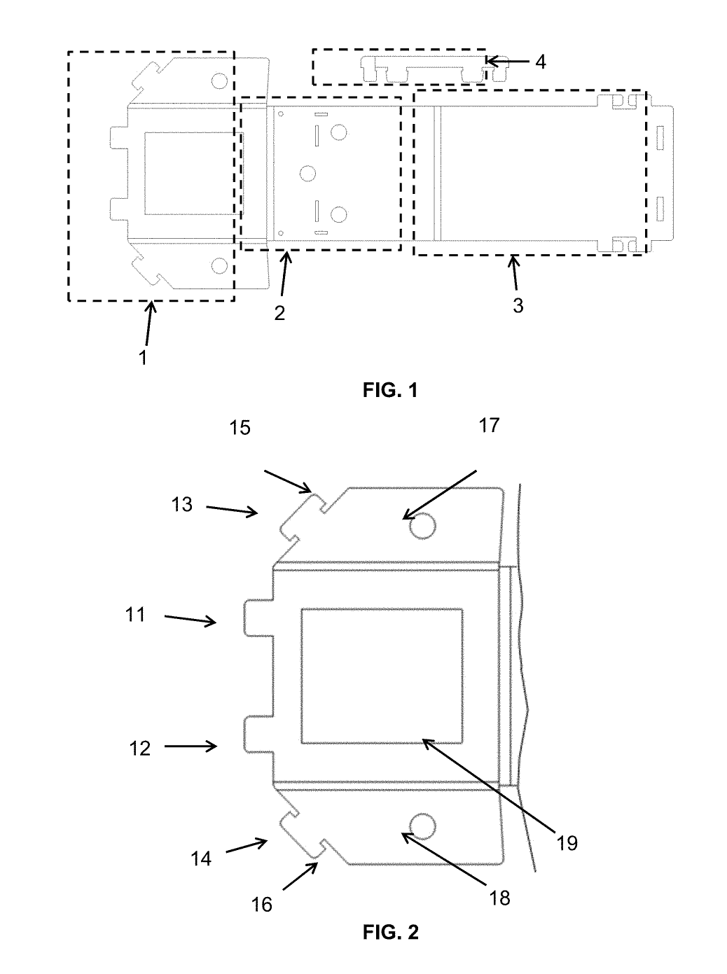

An example of the apparatus of the present invention is shown in FIGS. 1-5, wherein FIG. 1 shows a top view of the apparatus of the invention in a disassembled manner, wherein the first 1, second 2 and third 3 regions are separated by points of articulation of the same nature as the material itself and the support region 4 is individually implemented in additional material and, when the apparatus is assembled, said support region 4 is engaged with second region 2. In this example, the apparatus is implemented in reinforced cardboard-type material, thus allowing folding of its parts.

FIG. 2 shows a detailed view of the first region 1, showing its components, which are composed of two projections at the ends 11 and 12, a first projection 13 disposed on a first side support flap 15, a second projection 14 disposed on a second side support flap 16, side apertures 17 and 18 and an access window 19. The first side aperture 17 is arranged on the first side support flap 15 and the second side aperture 18 is arranged on the second side support flap 16, wherein such side apertures allow the user to practice the surgical procedure through a side angle, thus simulating a point commonly used during real laparoscopic procedures. Further, the access window 19 is arranged in order to allow incidence of ambient lighting, so that the user may carry out the desired training.

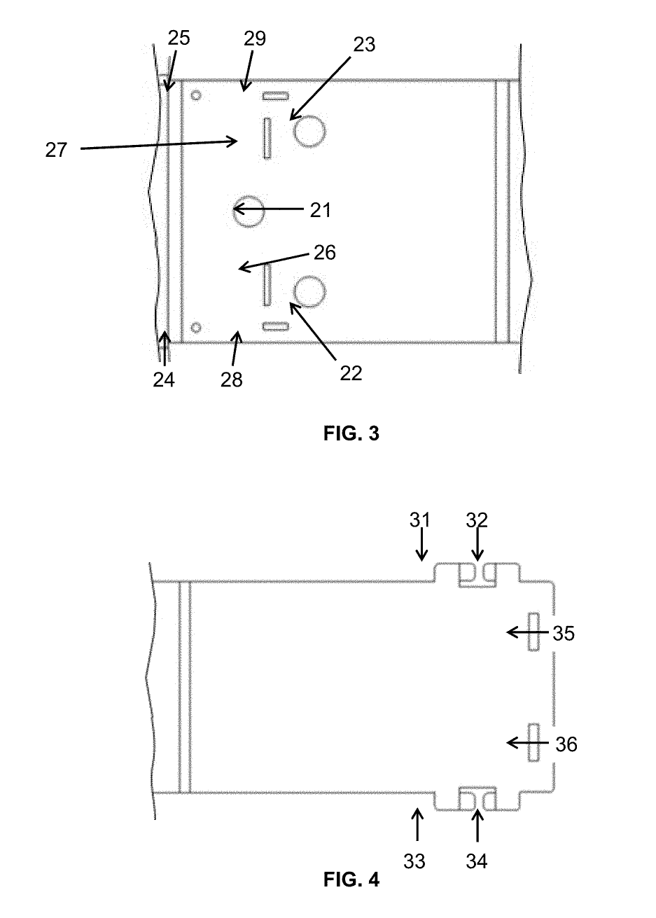

FIG. 3 shows a detailed view of the second region 2, in the present example, wherein the elements thereof are defined by an aperture for placing an image capture element 21, a first 22 and a second aperture 23 for insertion of medical equipment intended for training, a first 24 and a second aperture 25 for insertion of medical equipment at rest, i.e., equipment which are not being used, and a set of openings 26, 27, 28 and 29, intended for mounting and supporting the support region 4. The third opening 28 is perpendicular to the first opening 26 and the fourth opening 29 is perpendicular to the second opening 27, wherein said the fourth opening 29 is opposite to the third opening 28. Thus, as shown, the third opening 28 and the fourth opening 29 are arranged perpendicularly so as to allow the perpendicular projections of the support region 4 to be engaged.

FIG. 4 shows a detail view of the third region 3 showing its elements, namely, the side projections 31 and 32, the side projections 33 and 34 arranged opposite thereto, and the openings 35 and 36 arranged at the end.

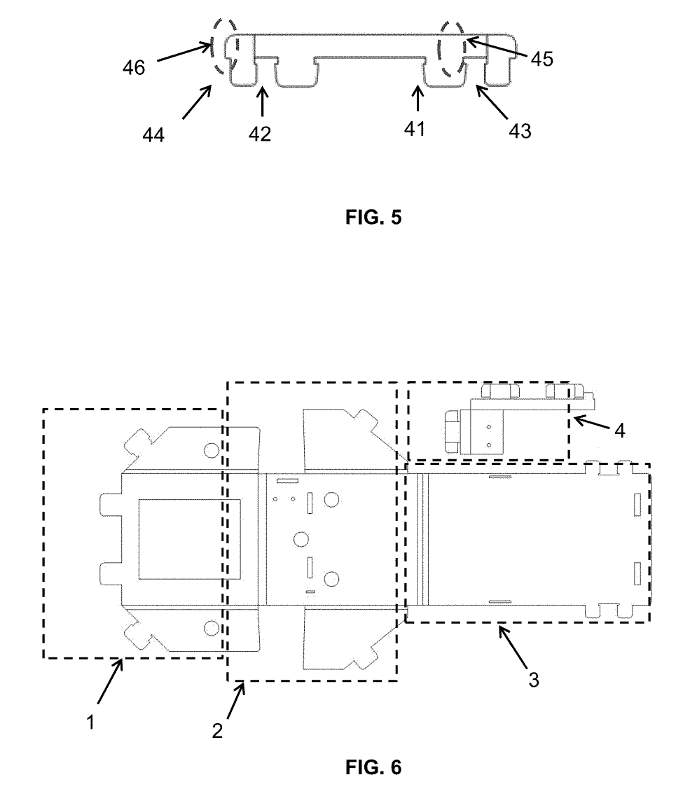

FIG. 5 shows a detail view of the support region 4 showing its components, namely, the projections 41, 42, 43 and 44. Between projections 41 and 43, and between projections 42 and 44, there is a point of articulation 45 and 46, of the same nature as the material itself, wherein folding at an angle, preferably 90°, between said projections 41 and 43 and between projections 42 and 44 is allowed. This allows for greater structural strength since, when the electronic device is placed in the support region 4, projections 43 and 44 are arranged in the same direction as the force applied by the device.

In order to assemble the apparatus, the first 1 and the third 3 regions are associated so that the projections at the ends 11 and 12 are connected to the openings 35 and 36 of the third region 3. The first projection 13 arranged on the first side support flap 15 is connected to a spacing between the side projections 31 and 32, the second projection 14, on a second side support flap 16, is connected to a spacing between the side projections 33 and 34.

Further, the support region 4 is associated with the second region 2, so that the first projection 41 of the support region 4 is introduced into the first opening 26 of the second region 2, the same occurring with the second projection 42, which is inserted into opening 27. As previously stated, the third projection 43 is folded at an angle, preferably 90°, with the first projection 41, and is thus introduced into the third opening 28, which in turn is arranged perpendicular to the first opening 26. The same also occurs with the fourth projection 44, inserted into the fourth opening 29. This fact provides a greater structural strength, which implies that the support region 4 can, thus, support greater weight.

### Example 2

Another exemplary apparatus of the present invention is shown in FIGS. 6-10, wherein FIG. 6 shows a top view of the apparatus of the invention in a disassembled manner, wherein the first 1, second 2 and third 3 regions are separated by points of articulation of the same nature as the material itself and the support region 4 is individually implemented in additional material and, when the apparatus is assembled, said support region 4 is engaged with second region 2. In this example, the second region 2 further comprises a support flap reinforcing the side support of the apparatus shown herein.

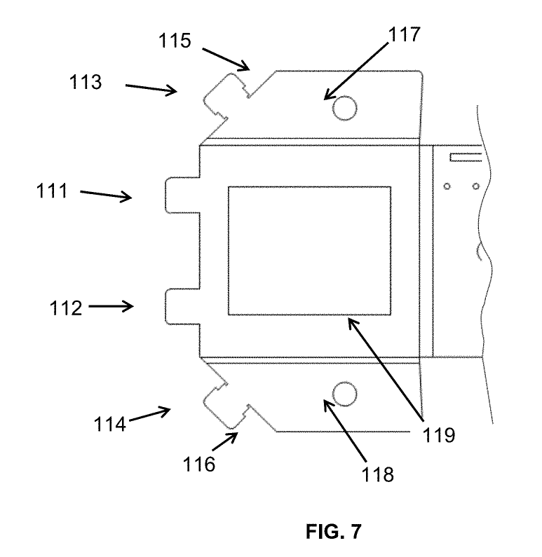

FIG. 7 shows a detailed view of the first region 1, showing its components, which are composed of two projections at the ends 111 and 112, a first projection 113 disposed on a first side support flap 115, a second projection 114 disposed on a second side support flap 116, side apertures 117 and 118 and an access window 119. The side aperture 117 is disposed on the first side support flap 115 and the second side aperture 118 is disposed on the second side support flap 116, wherein such side apertures allow the user to practice the surgical procedure through a side angle, thus simulating a point commonly used during real laparoscopic procedures. Further, the access window 119 is arranged in order to allow incidence of ambient lighting, so that the user may carry out the desired training.

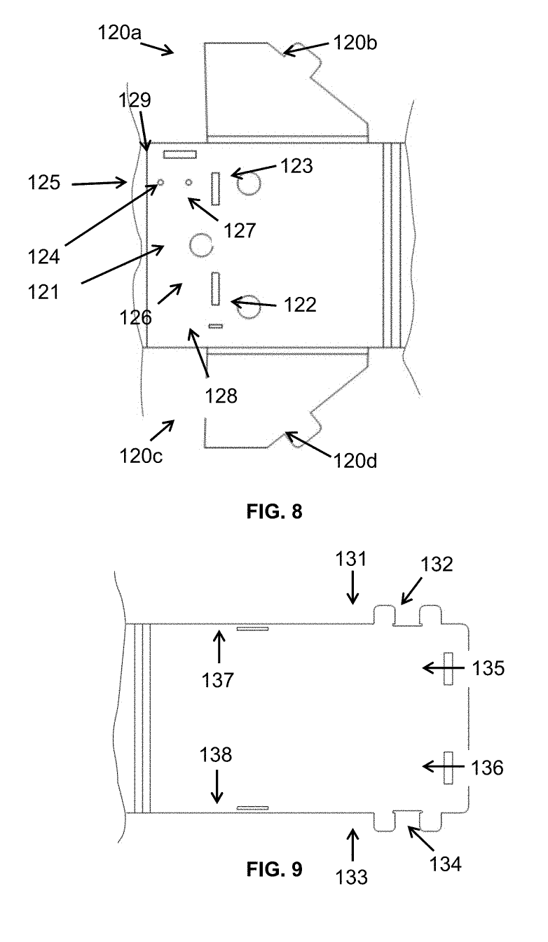

FIG. 8 shows a detailed view of the second region 2, in the present example, wherein the elements thereof are defined by an aperture for placing an image capture element 121, a first 122 and a second aperture 123 for insertion of medical equipment intended for training, a first 124 and a second aperture 125 for insertion of medical equipment at rest, i.e., equipment which are not being used, and a set of openings 126, 127, 128 and 129, intended for mounting and supporting the support region 4. The third opening 128 is perpendicular to the first opening 126 and the fourth opening 129 is perpendicular to the second opening 127, wherein said opening 129 is opposite to opening 128. Thus, as shown, the third opening 128 and the fourth opening 129 are arranged perpendicularly, wherein the fourth opening 129 is arranged displaced relative to the third opening 128, being asymmetric.

In this embodiment, the second region 2 further comprises two side support flaps, the first side support flap 120a comprising a first projection 120b opposite to the second side support flap 120c, which comprises a second projection 120d.

Further, FIG. 9 shows a detailed view of the third region 3 showing its components, namely, the side projections 131 and 132, the side projections 133 and 134 arranged opposite thereto, the openings 135 and 136 arranged at the end, and the openings 137 and 138 opposite to each other.

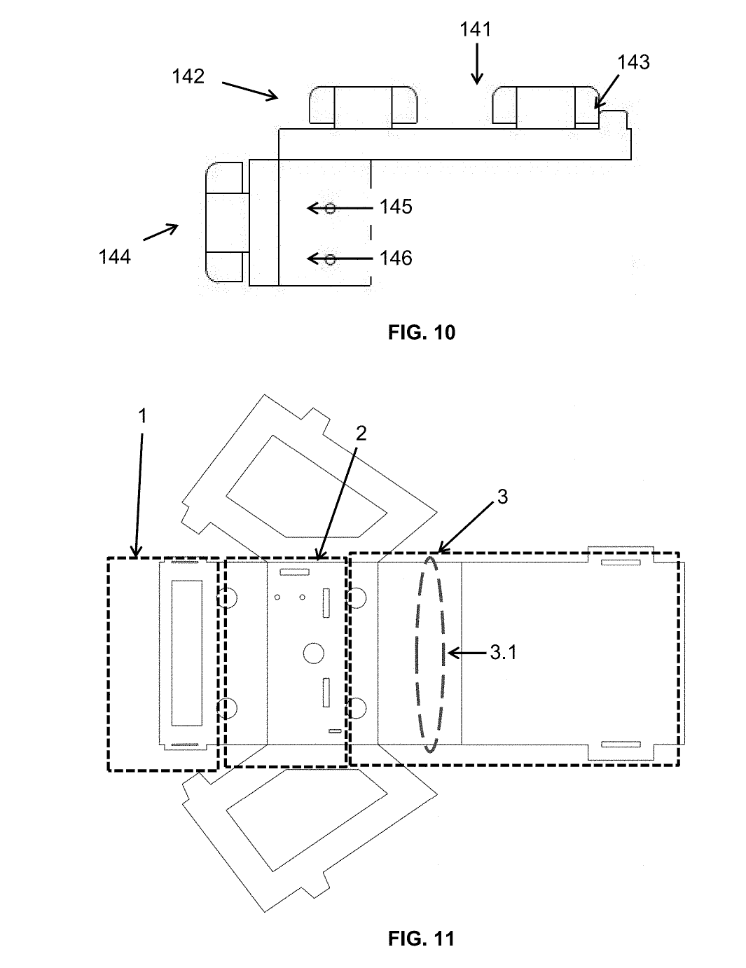

In FIG. 10, the support region 4 is shown with a detail of the parts thereof, the projections 141, 142, 143 and 144, the apertures 145 and 146 for inserting the resting surgery elements. Said apertures 145 and 146 are arranged to overlap the apertures 124 and 125 in the second region 2 at the time of assembly of the apparatus.

For assembling the apparatus, the association between the first 1 and the third 3 region is performed as shown in the abovementioned Example 1. In this example, however, the side support flaps of the second region 2 are folded at an angle of approximately 90°, and the projections 120b and 120d of the flaps are associated with the openings 137 and 138 of the third region 3.

### Example 3

In another exemplary apparatus of the present invention, FIG. 11 is shown, which is a top view of the apparatus of the invention in a disassembled manner, wherein the first 1, second 2 and third 3 regions are separated by points of articulation of the same nature as the material itself and the support region 4 is individually implemented in additional material and, when the apparatus is assembled, said support region 4 is engaged with second region 2. In this example, the second region 2 comprises two side support flaps.

The first region 1 comprises an aperture for incidence of ambient lighting, in addition to comprising upper apertures for introducing medical equipment, allowing training at varied angles, and also comprises openings at the side ends. The second region 2 is formed in the same manner as that shown in Example 2, but the side flap, in this case, comprises a window opening so as to allow the incidence of ambient lighting, in addition to comprising top and bottom projections, wherein, when the apparatus is assembled, the top projections engage with the end openings of the first region 1 and the bottom projections engage with the end openings of the third region 3.

Further, in this example, the third region 3 comprises a sectioned region 3.1, where it is divided into two parts, so that this region is formed by points of articulation implemented in the sheet material itself. This sectioning of the third region 3 allows the increasing of the structural strength of the apparatus of the present invention.

### Example 4

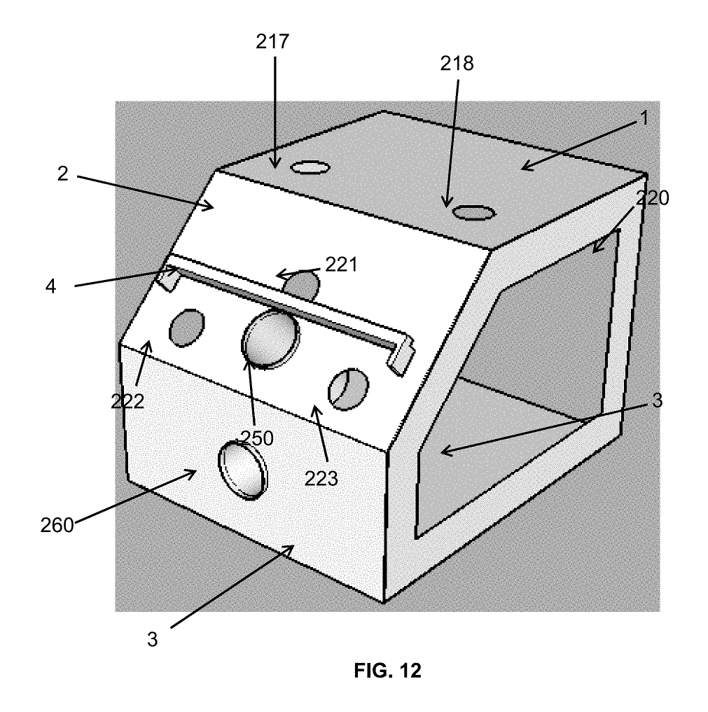

Another exemplary apparatus of the present invention is shown in FIGS. 12 to 15, wherein FIG. 12 shows a perspective view of the apparatus of the present invention, wherein the first 1, the second 2 and the third 3 regions are separated by points of articulation of the same nature as the material itself, and the support region 4 is individually implemented in additional material and, when the apparatus is assembled, said support region 4 engages with the second region 2.

The first region 1 comprises upper apertures for introducing medical equipment 217 and 218, allowing the user to carry out the training from another angle, simulating a particular type of surgical procedure. Further, the first region 1 comprises side end openings (not shown), as shown in Example 3, so as to allow connection with a side support flap 220 of the second region 2.

Accordingly, the second region 2 comprises an aperture for placing an image capture element 221, a first 222 and a second aperture 223 for introducing medical equipment, wherein said medical equipment relates to laparoscopic elements, two side support flaps 220, in addition to comprising an enlarged aperture 250 for practicing other types of surgical procedures, wherein, for example, apertures 222 and 223 are used for endoscopic laparoscopy procedures and the enlarged aperture 250 is used for performing gynecological laparoscopic procedures for transvaginal surgery.

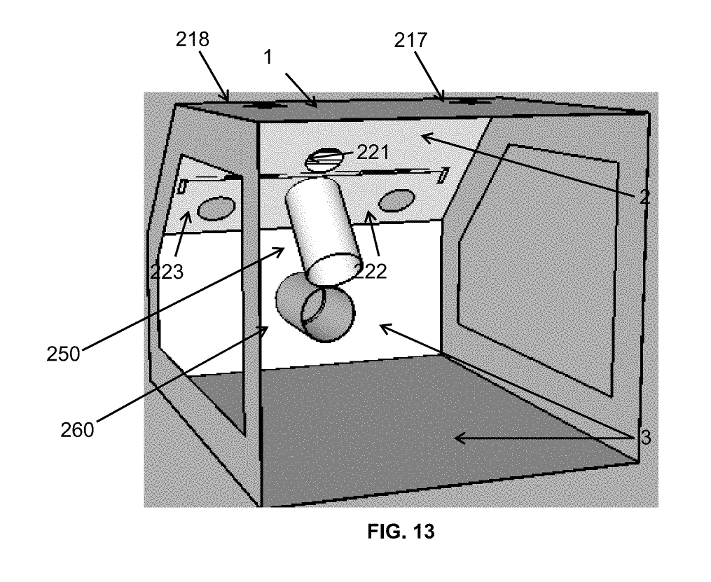

Further, in FIG. 13, where a rear perspective view of an example of the apparatus of the present invention is shown, it can be seen that the enlarged aperture 250 also comprises a cylindrical structure which enables simulating the actual transvaginal-type surgical procedure.

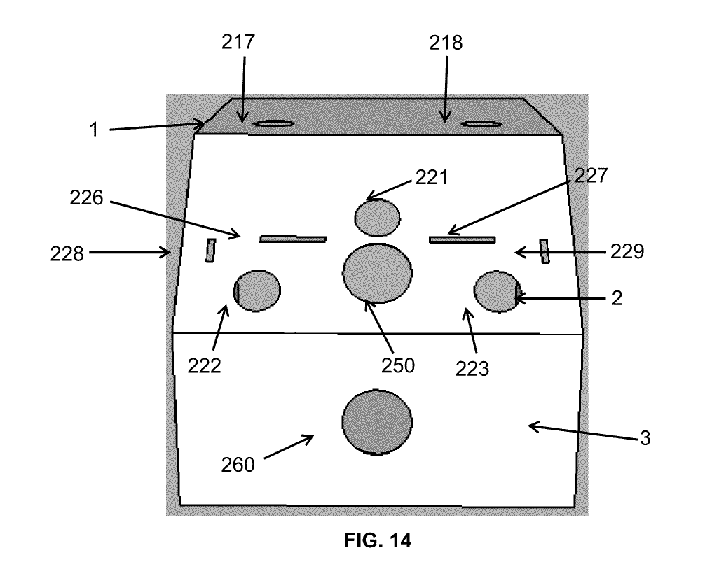

FIG. 14 shows a front view of the present example of the apparatus of the present invention, where the openings 226, 227, 228 and 229 are outlined, which enable insertion of the support region 4, so that the third 228 and the fourth 229 openings are arranged opposite to each other and perpendicular to the openings 226 and 227, whereby when the support region 4 is connected to the second region 2 the structural strength for the electronic device is improved, thus supporting greater force caused by said device.

Further, the third region 3 comprises a sectioned region as shown in Example 3 above, allowing the increasing of the structural strength of the apparatus of the present invention, where such point can be seen in FIG. 13. Notwithstanding, the third region 3 comprises an enlarged aperture 260 to allow surgical procedures to be performed, which has such angle, wherein the user inserts the laparoscopic equipment primarily perpendicularly to the third region 3 and perpendicularly to the ground or where the apparatus is positioned. Also in FIG. 13, it is shown that the enlarged aperture 260 comprises a cylindrical structure for channel simulation in a real surgical procedure, wherein this type of procedure is used in transanal laparoscopic surgery.

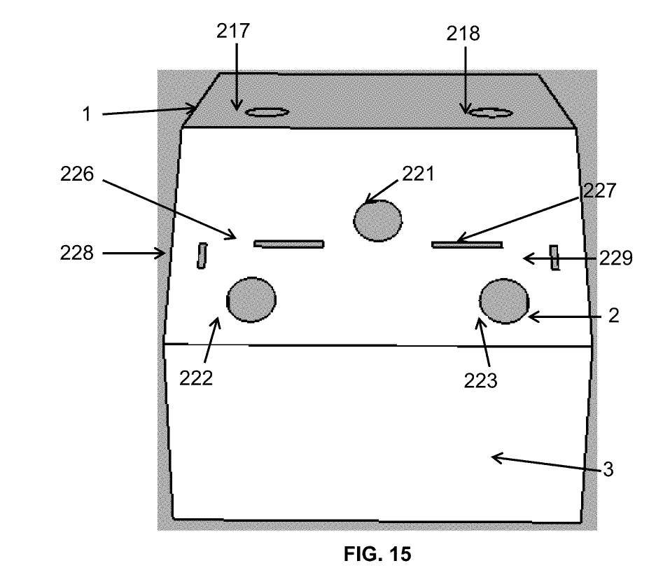

In a variation of this example, FIG. 15 shows the third region 3 without the enlarged aperture, so that said region is used only for purposes of ensuring structural strength to the apparatus of the present invention.

Those skilled in the art will appreciate the knowledge shown herein and may reproduce the invention in the modes shown and in other variants encompassed by the scope of the appended claims.

1. A surgical procedure training apparatus comprising:
a) a first region;
b) a second region;
c) a third region; and
d) a support region;
wherein,
said apparatus is implemented in continuous sheet material comprising points of articulation between: the first region and the second region; and the second region and the third region;
said points of articulation are of the same nature as the sheet material itself;
the second region comprises fastening means for connecting the support region; and
all said regions comprise fastening means for interconnecting one another.

2. The surgical procedure training apparatus according to claim 1, further comprising at least one support flap arranged either in the first region, the second region, or the third region by means of points of articulation.

3. The surgical procedure training apparatus according to claim 2, wherein the support flap comprises fastening means for connection with the other regions.

4. The surgical procedure training apparatus according to claim 1, wherein the fastening means are of the openings and projections type.

5. The surgical procedure training apparatus according to claim 1, wherein the first region comprises at least one aperture for introducing medical equipment.

6. The surgical procedure training apparatus according to claim 1, wherein the second region comprises at least one aperture for introducing medical equipment.

7. The surgical procedure training apparatus according to claim 6, wherein the second region comprises at least one aperture for image capture.

8. The surgical procedure training apparatus according to claim 1, wherein the support region is adapted to receive an electronic device having an image capture element.

9. The surgical procedure training apparatus according to claim 8, wherein the support region comprises at least one perpendicular projection when attached to at least one opening in the second region.

10. The surgical procedure training apparatus according to claim 1, wherein the second region comprises apertures for inserting medical equipment at rest.

11. The surgical procedure training apparatus according to claim 1, wherein the third region further comprises aperture for introducing medical equipment.

12. The surgical procedure training apparatus according to claim 1, further comprising at least one opening for ambient lighting and/or that sheet material comprises sufficient flexibility to allow the process of folding.

13. The surgical procedure training apparatus according to further comprising an aperture for introducing medical equipment that is adapted for laparoscopy-related surgical procedure devices, or for endoscopic laparoscopy, gynecological laparoscopy, transanal laparoscopy, or the like.

14. A laparoscopic procedure training system comprising:
a) a surgical procedure training apparatus;
b) trocars;
c) an electronic device having an image capture element;
d) a laparoscopic instrument; and
e) a training element;
wherein,
said surgical procedure training apparatus is provided with apertures for introducing medical equipment and apertures for placing an image capture element;
the laparoscopic instrument and the trocar are associated and introduced into the aperture for introducing the medical equipment of the apparatus;
the training element is inserted inside the apparatus; and
the electronic device is associated with the apparatus so that the capture element is associated with the aperture for placing the image capture element.

15. The laparoscopic procedure training system according to claim 14, wherein said surgical procedure training apparatus comprises:
a) a first region;
b) a second region;
c) a third region; and
d) a support region;
wherein
said apparatus is implemented in continuous sheet material comprising points of articulation between: the first region and the second region; and the second region and the third region;
said points of articulation are of the same nature as the sheet material itself;
the second region comprises fastening means for connecting the support region; and
all said regions comprise fastening means for interconnecting one another.
